(19) United States
(12) Patent Application Publication
(10) Pub. No.: US 2024/0402058 A1
(43) Pub. Date: Dec. 5, 2024

(54) SYSTEMS AND METHODS FOR PERFORMING A REAL-TIME ASSAY OF A SAMPLE

(71) Applicant: AMGEN INC., Thousand Oaks, CA (US)

(72) Inventor: Chao-Hsiang WU, Thousand Oaks, CA (US)

(73) Assignee: AMGEN INC., Thousand Oaks, CA (US)

(21) Appl. No.: 18/805,197

(22) Filed: Aug. 14, 2024

Related U.S. Application Data
(62) Division of application No. 16/635,941, filed on Jan. 31, 2020, now Pat. No. 12,104,995.
(60) Provisional application No. 62/539,803, filed on Aug. 1, 2017.

(51) Int. Cl. G01N 1/34; G01N 1/38; G01N 33/68

(52) U.S. Cl. CPC G01N 1/34; G01N 33/6848; G01N 2001/385

(57) ABSTRACT

Systems, and methods that facilitate the performance of an assay of a sample substantially in real-time. Thus, the assay can be performed, and the desired result obtained, much more quickly than allowed by conventional systems and methods.

### CROSS-REFERENCE TO RELATED APPLICATION

The present application claims the priority benefit of U.S. Provisional Patent Application No. 62/539,803, entitled “Systems and Methods for Performing a Real-Time Assay of a Sample” and filed Aug. 1, 2017, the entire disclosure of which is hereby incorporated by reference herein.

### FIELD OF THE DISCLOSURE

The present disclosure relates generally to assays and, more specifically, to performing a real-time assay of a sample.

### SEQUENCE LISTING

The present application is being filed with a sequence listing in electronic format. The sequence listing provided as a file titled, “51800_Seqlisting.txt” created Jul. 31, 2018 and is 263,964 bytes in size. The information in the electronic format of the sequence listing is incorporated herein by reference in its entirety.

### BACKGROUND

Assays are commonly performed to quantify one or more attributes of an analyte such as a drug, a biochemical substance, or a cell. An example of such an assay is the multi-attribute method (MAM) assay, which can detect and quantify Critical Quality Attributes (CQAs), identified by the Quality Target Product Profile (QTPP), of a sample (Development of a quantitative mass spectrometry multi-attribute method for characterization, quality control testing and disposition of biologics. Rogers RS, Nightlinger NS, Livingston B, Campbell P, Bailey R, Balland A. MAbs. 2015; 7(5): 881-90). The MAM assay is a manually-operated process that is performed in, for example, a Large Molecule Release Testing (LMRT) laboratory. MAM is a liquid chromatography (LC)—mass spectrometry (MS)-based peptide mapping method, comprising three steps: (1) sample preparation (such as polypeptide denaturation, reduction, alkylation, and digestion; (2) separation of the digested polypeptides by LC and detection by MS; and (3) analysis of the data for targeted CQAs and detection of new signal (i.e., peaks) when compared to a reference standard.

CQAs are chemical, physical, or biological properties that are present within a specific value or range values. For example, for large polypeptide therapeutic molecules, physical attributes and modifications of amino acids (the building blocks of polypeptides) are important CQAs that are monitored during and after manufacturing, as well as during drug development. Unlike conventional analytical assays that track changes in peak size and peak shape of whole or partial polypeptides, MAM detects specific CQAs at the amino acid level.

However, while MAM is an important advance in the assessment of CQAs of polypeptide therapeutic molecules during development, manufacturing, and storage (assessing, e.g., stability), analysis can consume seven to ten days, from sample preparation to final analysis, time that drives costs and delays development and drug release. For example, polypeptide therapeutics are customarily produced by cultured cells expressing the target polypeptide. Such production systems are not easily “put on hold” while MAM analysis of CQAs is pending, resulting in the cells producing the target polypeptide with CQAs that do not meet specifications—a waste of time, materials, and manpower. Furthermore, delays of seven to ten days accumulate during drug development, for example, when optimizing culture conditions, impeding delivery of important and new polypeptide pharmaceuticals to patients. Thus, there is a need for efficient and faster methods to facilitate CQA analysis using MAM.

### SUMMARY

One aspect of the present disclosure provides a method for performing a real-time assay. The method includes the steps of: (a) moving a sample of a product containing polypeptides to a polypeptide-binding column via a first holding coil; (b) binding the polypeptides in the sample to the polypeptide binding column, thereby separating the polypeptides in the sample from a remainder of the sample; (c) moving an elution buffer solution from a buffer source to the polypeptide binding column, via the first holding coil, and through a second holding coil downstream of the polypeptide binding column, thereby eluting polypeptides bound to the polypeptide binding column and moving an elution/polypeptide mixture comprising the elution buffer solution and the eluted polypeptides to the second holding coil; (d) moving the elution/polypeptide mixture from the second holding coil to a reaction chamber; (c) incubating the polypeptides in the elution/polypeptide mixture in the reaction chamber, resulting in denatured polypeptides; (f) moving a reducing reagent that cleaves disulfide bond crosslinks to the reaction chamber via the first holding coil after (e); (g) incubating the denatured polypeptides with the reducing reagent in the reaction chamber, resulting in denatured and reduced polypeptides; (h) moving an alkylating reagent that alkylates sulfhydryls to the reaction chamber after (g); (i) incubating the denatured and reduced polypeptides with the alkylating reagent in the reaction chamber, thereby alkylating the denatured and reduced polypeptides; (j) moving the denatured, reduced, and alkylated polypeptides, the elution buffer solution, the reducing reagent, and the alkylating reagent from the reaction chamber to a desalting column via the first holding coil, the desalting column equilibrated with a proteolysis buffer; (k) applying the denatured, reduced, and alkylated polypeptides to the desalting column, thereby separating the denatured, reduced, and alkylated polypeptides from the reducing and alkylating reagents, and resulting in desalted polypeptides; (l) moving the desalted polypeptides to a proteolytic enzyme column downstream of the desalting column; (m) digesting the desalted polypeptides in the third polypeptide column, resulting in digested polypeptides; and (n) moving the digested polypeptides to an analytical device for analysis of the digested polypeptides.

Another aspect of the present disclosure provides a method for performing a real-time assay using a closed system including a multi-port valve, a first holding coil upstream of the multi-port valve, a polypeptide binding column fluidly coupled to and downstream of a first port of the multi-port valve, a second holding coil fluidly coupled to and downstream of the polypeptide binding column, a reaction chamber fluidly coupled to and downstream of second and third ports of the multi-port valve, a desalting column fluidly coupled to and downstream of a fourth port of the multi-port valve, and a proteolytic enzyme column downstream of the desalting column. The method includes: (a) moving, via a controller communicatively coupled to the closed system, a sample of a product containing polypeptides to the first holding coil; (b) positioning, via the controller, the multi-port valve in a first position in which the first holding coil is connected to the polypeptide binding column via the first port of the multi-port valve, such that the sample flows to the first column, whereby polypeptides in the sample bind to the polypeptide binding column; (c) when the multi-port valve is in the first position, moving, via the controller, an elution buffer solution from a source of elution buffer solution to the second holding coil via the polypeptide binding column, such that the elution buffer solution elutes substantially all of the polypeptides bound to the polypeptide binding column; (d) moving, via the controller, the multi-port valve to a second position in which the second holding coil is connected to the reaction chamber via the second port of the multi-port valve; (e) when the multi-port valve is in the second position, moving, via the controller, an elution/polypeptide mixture comprising the elution buffer solution and the eluted polypeptides to the reaction chamber, whereby the polypeptides in the elution/polypeptide mixture are denatured; (f) moving, via the controller, the multi-port valve to a third position in which the first holding coil is connected to the reaction chamber via the third port of the multi-port valve; (g) after (f), moving, via the controller, a reducing reagent that cleaves disulfide bond crosslinks to the first holding coil, and moving, via the controller, the reducing reagent from the first holding coil to the reaction chamber via the third port of the multi-port valve, thereby reducing the denatured polypeptides; (h) after (g), moving, via the controller, an alkylating reagent that alkylates sulfhydryl groups to the first holding coil, and moving, via the controller, the alkylating reagent from the first holding coil to the reaction chamber via the third port, thereby alkylating the denatured and reduced polypeptides; (i) moving, via the controller, the alkylated polypeptides, the elution buffer solution, the reducing reagent, and the alkylating reagent from the reaction chamber to the first holding coil via the third port; (j) moving, via the controller, the multi-port valve to a fourth position in which the first holding coil is connected to the desalting column via the fourth port of the multi-port valve, and, when the multi-port valve is in the fourth position, moving the alkylated polypeptides, the elution buffer solution, the reducing reagent, and the alkylating reagent from the first holding coil to the desalting column, whereby the denatured, reduced, and alkylated polypeptides are applied to the desalting column, thus separating the denatured, reduced, and alkylated polypeptides from the reducing and alkylating reagents, resulting in desalted polypeptides; (k) moving, via the controller, the desalted polypeptides to the proteolytic enzyme column, whereby the desalted polypeptides are digested; and (l) passing, via the controller, the digested polypeptides to an analytical device, whereby the digested polypeptides are analyzed.

Another aspect of the present disclosure provides a closed system for performing an online, real-time assay. The system includes: a first holding coil fluidly arranged to receive a sample of a product containing polypeptides; a multi-port valve fluidly coupled to and located downstream of the first holding coil; a polypeptide binding column fluidly coupled to the multi-port valve and arranged to receive the sample from the first holding coil via a first port of the multi-port valve, the polypeptide binding column configured to bind the polypeptides from the sample; a buffer source fluidly coupled to the multi-port valve and arranged to supply elution buffer solution to a second holding coil located downstream of the polypeptide binding column, such that the elution buffer solution is adapted to elute substantially all of the polypeptides from the polypeptide binding column; a reaction chamber fluidly coupled to the multi-port valve and arranged downstream of the polypeptide binding column, the reaction chamber adapted to receive a mixture from the polypeptide binding column via a second port of the multi-port valve, the mixture comprising the elution buffer solution and the eluted polypeptides, wherein the polypeptides of the mixture are denatured in the reaction chamber, wherein the reaction chamber is arranged to receive a reducing reagent that cleaves disulfide bond crosslinks via the first holding coil and a third port of the multi-port valve, the reducing reagent reduces the denatured polypeptides, and wherein the reaction chamber is further arranged to receive an alkylating reagent that alkylates sulfhydryls via the first holding coil and the third port of the multi-port valve, wherein the alkylating reagent alkylates the denatured and reduced polypeptides in the reaction chamber; a desalting column fluidly coupled to the multi-port valve and arranged to receive the denatured, reduced, and alkylated polypeptides, the elution buffer solution, and the alkylating reagent from the reaction chamber, the desalting column configured to separate the denatured, reduced, and alkylated polypeptides from the elution buffer solution, the reducing reagent, and the alkylating reagent; and a proteolytic enzyme column fluidly coupled to and arranged downstream of the second polypeptide column to obtain the separated polypeptides from the desalting column, the proteolytic enzyme column configured to digest the desalted polypeptides.

Another aspect of the present disclosure provides a closed system for performing a real-time assay. The system includes: a multi-port valve; a first holding coil upstream of the multi-port valve; a polypeptide binding column fluidly coupled to and downstream of a first port of the multi-port valve; a second holding coil fluidly coupled to and downstream of the polypeptide binding column; a reaction chamber fluidly coupled to and downstream of second and third ports of the multi-port valve; a desalting column fluidly coupled to and downstream of a fourth port of the multi-port valve; a proteolytic enzyme column downstream of the desalting column; and a controller communicatively coupled to the multi-port valve. The controller includes a memory, a processor, and logic stored on the memory and executable by the processor to: (a) move a sample of a product containing polypeptides to the first holding coil; (b) position the multi-port valve in a first position in which the first holding coil is connected to the polypeptide binding column via the first port of the multi-port valve, such that the sample flows to the first column, whereby polypeptides in the sample bind to the polypeptide binding column; (c) when the multi-port valve is in the first position, move an elution buffer solution from a source of elution buffer solution to the second holding coil via the polypeptide binding column, such that the elution buffer solution elutes substantially all of the polypeptides bound to the polypeptide binding column; (d) move the multi-port valve to a second position in which the second holding coil is connected to the reaction chamber via the second port of the multi-port valve; (e) when the multi-port valve is in the second position, move an elution/polypeptide mixture comprising the elution buffer solution and the eluted polypeptides to the reaction chamber, whereby the polypeptides in the elution/polypeptide mixture are denatured; (f) move the multi-port valve to a third position in which the first holding coil is connected to the reaction chamber via the third port of the multi-port valve; (g) after (f), move a reducing reagent that cleaves disulfide bonds to the first holding coil, and move the reducing reagent from the first holding coil to the reaction chamber via the third port of the multi-port valve, thereby reducing the denatured polypeptides; (h) after (g), move an alkylating reagent that alkylates sulfhydryls to the first holding coil, and move the alkylating reagent from the first holding coil to the reaction chamber via the third port, thereby alkylating the denatured and reduced polypeptides; (i) move the denatured, reduced, and alkylated polypeptides, the elution buffer solution, the reducing reagent, and the alkylating reagent from the reaction chamber to the first holding coil via the third port; (j) move the multi-port valve to a fourth position in which the first holding coil is connected to the desalting column via the fourth port of the multi-port valve, and, when the multi-port valve is in the fourth position, move the denatured, reduced, and alkylated polypeptides, the elution buffer solution, the reducing reagent, and the alkylating reagent from the first holding coil to the desalting column, whereby the denatured, reduced, and alkylated polypeptides are de-salted; (k) move the desalted polypeptides to the proteolytic enzyme column, whereby the desalted polypeptides are digested; and (l) pass the digested polypeptides to a glycan analysis device, whereby the digested polypeptides are separated and quantified.

### BRIEF DESCRIPTION OF THE DRAWINGS

FIG. 1 is a schematic diagram of a system for performing an online, real-time assay assembled in accordance with the teachings of the present disclosure.

FIG. 2 is a schematic diagram of a controller of the system illustrated in FIG. 1.

FIGS. 3A and 3B are graphs depicting the results of a study monitoring the effectiveness of the system of FIG. 1 over a production run of 40 days.

FIG. 3C is a graph depicting a snapshot of the results of FIG. 3B over a 8 day period of time.

### DETAILED DESCRIPTION OF THE PREFERRED EMBODIMENTS

The present disclosure provides for real-time assays and methods that allows for the monitoring and control of CQAs in real-time, so that the desired final polypeptide therapeutic product can be produced. The disclosed assays and methods can facilitate MAM result turn-around time such that results are available within a few hours (e.g., two to three hours) instead of a typical, manually operated, off-line measurement result of seven to ten days. Thus, the disclosed assays and methods improve the turn-around time by approximately 54-fold to 120-fold. Such “on-the-fly” (real time) results enable, for example, adjusting production variables to manufacture efficiently (online) products having appropriate CQAs.

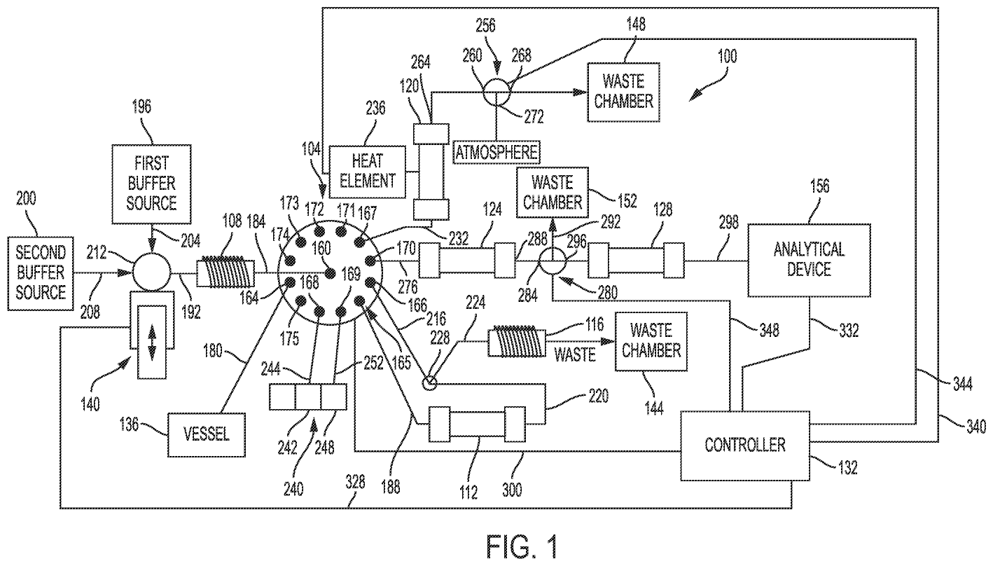

FIG. 1 illustrates a schematic diagram of a system 100 assembled in accordance with the teachings of the present disclosure. The system 100, which can be located at or in a laboratory (e.g., a Large Molecule Release Testing laboratory) is a closed system for automatically or substantially automatically performing an assay of a sample of a product containing polypeptides, as is described in greater detail below. By automating (or substantially automating) this process using the system 100, the assay can be performed in real-time (or substantially in real-time, such that the entire process can be performed, and the desired result obtained, in a matter of hours (e.g., 2 to 3 hours), a significant improvement over the 7 to 10 days typically required by the conventionally known, manually-operated MAM assays. Moreover, the closed nature of the process that utilizes the system 100 maintains sterile conditions.

In this version, the polypeptides can be therapeutic polypeptides, which are discussed further below

The system 100 illustrated in FIG. 1 generally includes a multi-port valve 104, a first holding coil 108, a first column 112, a second holding coil 116, a reaction chamber 120, a second column 124, a third column 128, and a controller 132. In the version illustrated in FIG. 1, the system 100 also includes a vessel 136, a pump 140, a first waste chamber 144, a second waste chamber 148, a third waste chamber 152, and an analytical device 156 for analyzing the polypeptides. In other versions, however, the system 100 can not include one or more of these components. As an example, the system 100 can not include the vessel 136 and/or the analytical device 156. In any event, generally conventional plumbing extends between each of the components of the system 100 so as to facilitate fluid communication between components of the system 100 when desired, as is described in greater detail below. The multi-port valve 104 is generally configured to control fluid communication between the various components of the system 100. In this version, the multi-port valve 104 is a twelve satellite port and a central shared port valve. In other words, the multi-port valve 104 has a central port 160 and twelve satellite ports 164-175 that are selectively fluidly coupled to the central port 160. The multi-port valve 104 is movable between twelve different positions that fluidly couple the central port 160 with the twelve different satellite ports 164-175, respectively (some of which are not utilized in the operation of the system 100 of FIG. 1). In other versions, the multi-port valve 104 can include more or less satellite ports, can be a different type of valve or can be replaced by one or more different valves (each having one or more ports). As an example, the multi-port valve 104 can be replaced by a plurality of single port valves separately connected to and controlled by the controller 132, with each of the single port valves effectively replacing one of the satellite ports 164-175.

The vessel 136 is generally configured to hold or store the product containing polypeptides that is to be assayed. The vessel 136 in this version takes the form of a bioreactor that holds or stores the product. In other versions, however, the vessel 136 can instead take the form of a cell culture vessel, such as a flask, a plate, etc. The vessel 136 is fluidly coupled to the satellite port 164 of the valve 104 via a conduit 180 of the plumbing, such that the valve 104 can, when desired, obtain a sample of the product contained in the vessel 136 from the vessel 136.

The first holding coil 108 is located upstream of the valve 104 and is fluidly coupled to the central port 160 of the valve 104 via a conduit 184 of the plumbing. The first holding coil 108 is thus arranged to receive the sample of the product from the vessel 136, via the valve 104, when the valve 104 is in a first position in which the central port 160 is fluidly coupled to the satellite port 164.

The first column 112 is located downstream of the valve 104 and is fluidly coupled to the satellite port 165 of the valve 104 via a conduit 188 of the plumbing. The first column 112 is thus arranged to receive the sample from the first holding coil 108, via the valve 104, when the valve 104 is in a second position in which the central port 160 is fluidly coupled to the satellite port 165. When the first column 112 receives the sample, the first column 112 is configured to bind polypeptides from the sample as the sample flows therethrough. In this manner, the first column 112 separates the polypeptides in the sample from a remainder of the sample, which can be passed to the first waste chamber 144 via the second holding coil 116.

The first column 112, which can also be referred to herein as a polypeptide-binding column, is selected from the group consisting of a protein A column, a protein G column, a protein A/G column, a protein L column, an amino acid column, an avidin column, a streptavidin column, a carbohydrate bonding column, a carbohydrate column, a glutathione column, a heparin column, a hydrophobic interaction column, an immunoaffinity column, a nucleotide/coenzyme column, a specialty column, and an immobilized-metal affinity chromatography (IMAC) column. For example, in the case of polypeptides that are human IgGs of subclasses 1, 2, or 4, IgM, IgA, or IgE (and comprising a human Fc portion and/or a Fab region of the human VH3 family), protein A columns are useful. Protein G columns can be used to purify human IgGs of subclasses 1-4. Recombinant fusion protein A/G columns can also be used to purify all of these classes of human antibodies, as the fusion protein provides protein A and protein G binding sites. Thus, protein A/G fusion proteins can be used to purify human IgG, IgA, IgE, and IgM. Furthermore, protein L columns can be used to purify human IgG, IgM, IgA, IgE and IgD, provided the target antibodies have an appropriate kappa (κ) subtype light chain (i.e., VκI, VκIII and VκIV subtypes); protein L columns can also be used to purify Fab and scFv fragments also having the appropriate κ chain subtype, as protein L binds the variable (V) chain of antibodies.

The pump 140 is located upstream of and fluidly coupled to the first holding coil 108 via a conduit 192 of the plumbing. The pump 140 in this version takes the form of a syringe pump that is fluidly coupled to both a first buffer source 196 and a second buffer source 200. In this version, the first buffer source 196 is a elution buffer source that can supply an elution buffer solution 204, e.g., in the case of protein A-bound antibodies, an acidic buffer; or in the case of protein G-bound antibodies, very acidic (pH 3 or less) buffer; one of skill in the art is able to optimize and select appropriate elution buffers for bound antibodies, to the pump 140 (and, ultimately, the first holding coil 108), and the second buffer source 200 is a denaturing buffer (containing a denaturant that disrupts quaternary, tertiary, or secondary polypeptide structure) source that can supply a denaturing reagent 208 to the pump 140 (and, ultimately, the first holding coil 108). In other versions, the pump 140 can be a different type of pump and/or different pumps 140 can be used for each of the buffer sources 196, 200.

In some versions, the denaturing reagent can be or include a denaturing detergent or a chaotrope. In those versions in which the denaturing reagent is or includes a denaturing detergent, the denaturing detergent is preferably selected from the group consisting of sodium dodecyl sulfate (SDS), sodium cholate, sodium deoxycholate, sodium glycocholate, sodium taurocholate, sodium taurodeoxycholate, N-lauroylsarcosine, lithium dodecyl sulfate, hexadecyltrimethyl ammonium bromide (CTAB) and trimethyl (tetradecyl) ammonium bromide (TTAB). More preferably, the denaturing detergent is SDS. In those versions in which the denaturing reagent is or includes a chaotrope, the chaotrope is preferably selected from the group consisting of urea, n-butanol, ethanol, guanidinium chloride, lithium perchlorate, lithium acetate, magnesium chloride, phenol, 2-propanol, and thiourea. Alternatively or additionally, the denaturing reagent can be or include a heated fluid that has a temperature suitable for reaching, if not maintaining, a pre-determined temperature (e.g., about 22° C. to about 120° C.) in the reaction chamber 120 when the denaturing reagent is passed to the reaction chamber 120.

A valve 212 is located between the pump 140 and the first and second buffer sources 196, 200 to selectively fluidly couple the pump 140 to only one of the buffer sources 196, 200 at a time. More particularly, the valve 212 is movable between a first position, in which the pump 140 is fluidly coupled to the first buffer source 196 and the pump 140 is fluidly isolated from the second buffer source 200, and a second position, in which the pump 140 is fluidly coupled to the second buffer source 200 and the pump 140 is fluidly isolated from the first buffer source 196. In other words, the pump 140 is selectively fluidly coupled to the first buffer source 196 or the second buffer source 200 depending upon the position of the valve 212.

As noted above, the pump 140 in this version is a syringe pump. The syringe pump 140 is generally configured to obtain a buffer from one of the buffer sources 196, 200 and output that buffer to the first holding coil 108. When the valve 212 is in the first position, the pump 140 can obtain (e.g., draw in) the elution buffer solution 204 from the first buffer source 196, and, when desired, can output (e.g., eject) that elution buffer solution 204 to the first holding coil 108. Conversely, when the valve 212 is in the second position, the pump 140 can obtain (e.g., draw in) the denaturing reagent 208 from the second buffer source 200, and, when desired, can output (e.g., eject) that denaturing reagent 208 to the first holding coil 108.

The second holding coil 116 is located downstream of the valve 104, the first holding coil 108, and the first column 112. The second holding coil 116 is selectively fluidly coupled to the satellite port 166 of the valve 104 via conduits 216, 220 of the plumbing. The second holding coil 116 is also selectively fluidly coupled to the first column 112 via the conduit 220 and a conduit 224. A three-way valve 228 is arranged between the conduits 216, 220, 224 to facilitate the selective coupling in order to produce the desired fluid flow, as is described in greater detail below.

The second holding coil 116 is thus arranged to receive the elution buffer solution 204 from the first holding coil 108, via the valve 104 and through the first column 112, when the pump 140 outputs the elution buffer solution 204 in the manner described above, the valve 104 is in the second position in which the central port 160 is fluidly coupled to the satellite port 165. As the elution buffer solution 204 passes through the first column 112, the elution buffer solution 204 elutes substantially all of the polypeptides bound to the first column 112. The three-way valve 228 is operated to connect the conduits 220, 224, thereby fluidly coupling the first column 112 with the second holding coil 116, such that an elution/polypeptide mixture including the elution buffer solution 204 and the eluted polypeptides flows from the first column 112 to the second holding coil 116 via the conduits 220, 224.

The reaction chamber 120 is located downstream of the valve 104 and the first column 112, and is fluidly coupled to the satellite port 167 of the valve 104 via conduit 232 of the plumbing. In this version, the reaction chamber 120 is partially, if not completely, pre-filled with the denaturing reagent 208 (i.e., filled prior to operation of the system 100) from the second buffer source 200 by using the pump 140 to output the denaturing reagent 208 to the first holding coil 108 and moving the multi-port valve 104 to a fourth position in which the central port 160 is fluidly coupled to the satellite port 167 of the valve 167, thereby facilitating movement of the denaturing reagent 208 from the first holding coil 108 to the reaction chamber 120. In other versions, however, the reaction chamber 120 can be partially or completely filled with the denaturing reagent 208 during operation of the system 100 (e.g., after the second holding coil 116 receives the elution buffer solution 204), partially or completely filled with a denaturing reagent from another buffer source and/or in a different manner, or the reaction chamber 120 may not be filled at all, in which case heat can be applied to the reaction chamber 120 by a heating element 236 connected to the reaction chamber 120.

After the elution/polypeptide mixture reaches the second holding coil 116, the elution/polypeptide mixture is moved to the reaction chamber 120. In this version, the reaction chamber 120 indirectly receives the elution/polypeptide mixture from the second holding coil 116. More particularly, the three-way valve 228 is operated to connect the conduits 216, 224, the elution/polypeptide mixture is moved from the second holding coil 116 to the satellite port 166 of the multi-port valve 104 via the conduits 216, 224, the valve 104 is moved to a third position in which the central port 160 is fluidly coupled to the satellite port 166, such that the mixture moves to the first holding coil 108, and the valve 104 is moved to a fourth position in which the central port 160 is fluidly coupled to the satellite port 167 of the valve 104, such that the mixture moves from the first holding coil 108 to the reaction chamber 120. In other versions, the reaction chamber 120 can directly receive the elution/polypeptide mixture from the second holding coil 116 or can indirectly receive the mixture from the second holding coil 116 utilizing one or more different components and/or in a different order.

In any case, once the elution/polypeptide mixture reaches the reaction chamber 120, the polypeptides in the mixture incubate in the reaction chamber 120, thereby denaturing the polypeptides in the reaction chamber 120. When, for example, the reaction chamber 120 is at least partially filled with the denaturing reagent, the polypeptides in the mixture will, upon reaching the reaction chamber 120 and reacting with the denaturing reagent, undergo denaturation. In some cases, the reaction chamber 120 can, at the same time or at all times, be heated by the heating element 236 connected to (e.g., positioned immediately adjacent, surrounding) the reaction chamber 120 to help facilitate the denaturation process. In other words, the heating clement 236 can apply heat, preferably heat having a temperature of about 22° C. to about 120° C. and, more preferably, heat having a temperature of about 40° C., to the reaction chamber 120 to encourage denaturation. The heating element 236 can, for example, take the form of a heating block, a heating coil, an induction heater, a heat pump, a cartridge heater, an electrical resistance wire, a heated fluid, or other element suitable for heating one or more portions of the reaction chamber 120. In any event, by applying heat from the heating element 236 to the reaction chamber 120, the process of denaturation can be facilitated. In other cases, however, the reaction chamber 120 can not be filled with the denaturing reagent and the denaturation process can be facilitated only by applying heat from the heating clement 236.

Following the denaturation of the polypeptides in the mixture, the reaction chamber 120 is configured to receive a reducing reagent that cleaves disulfide bond crosslinks, thereby reducing the denatured polypeptides. The reducing reagent can be selected from the group consisting of dithiothreitol (DTT), glutathione, β-mercaptoethanol (β-ME), and tris(2-carboxyethyl) phosphine (TCEP) and is generally supplied by cooling vessel 240, which can, for example, take the form of a chiller having a temperature of 4° C. In this version, the cooling vessel 240 is located downstream of the valve 104 and has a first chamber 242 that contains the reducing reagent and is fluidly coupled to the satellite port 168 of the valve 104 via conduit 244 of the plumbing. The first chamber 242 supplies the reducing reagent to the first holding coil 108 when the valve 104 is in a fifth position in which the central port 160 is fluidly coupled to the satellite port 168 of the valve 104, and the reducing reagent is then moved from the first holding coil 108 to the reaction chamber 120 when the valve 104 is in (or returns to) the fourth position (in which the central port 160 is fluidly coupled to the satellite port 167). Thus, in this version, the reducing reagent is indirectly supplied to the reaction chamber 120 via the first holding coil 108. In other versions, however, the first chamber 242 can directly supply the reducing reagent to the reaction chamber 120 (i.e., without moving the reducing reagent to the first holding coil 108).

After the denatured polypeptides are reduced, the reaction chamber 120 is configured to receive an alkylating agent that alkylates sulfhydryls in the reaction chamber 120, thereby alkylating the denatured and reduced polypeptides in the reaction chamber 120. The alkylating agent is preferably an alkylating reagent such as indole-3-acetic acid (IAA), though other alkylating agents can be used. In this version, the cooling vessel 240 has a second chamber 248 that contains the alkylating agent and is fluidly coupled to the satellite port 169 of the valve 104 via conduit 252 of the plumbing. The second chamber 248 supplies the alkylating agent to the first holding coil 108 when the valve 104 is in a sixth position in which the central port 160 is fluidly coupled to the satellite port 169 of the valve 104, and the alkylating agent is then moved from the first holding coil 108 to the reaction chamber 120 when the valve 104 is in (or returns) to the fourth position. Thus, in this version, the alkylating agent is indirectly supplied to the reaction chamber 120 via the first holding coil 108. In other versions, however, the second chamber 248 can directly supply the alkylating agent to the reaction chamber 120 (i.e., without moving the alkylating agent to the first holding coil 108) and/or the alkylating agent can be supplied from a different cooling vessel (e.g., a cooling vessel separate from the cooling vessel 240).

In this version, the system 100 further includes a first normally open valve 256 that is fluidly coupled to and located downstream of the reaction chamber 120. The normally open valve 256 has an inlet port 260 that is fluidly connected to an outlet 264 of the reaction chamber 120, a first outlet port 268 that is fluidly connected to the second waste chamber 148, and a second outlet port 272 that is fluidly connected to atmosphere. The normally open valve 256 normally operates in an open, or first, position when the system 100 is in operation and, more particularly, the reaction chamber 120 is receiving and being filled with the elution/polypeptide mixture, the reducing reagent, the alkylating agent, and, in some cases, the denaturing reagent, the valve 256. In this open position, the inlet port 260 is fluidly coupled to the first outlet port 268 and the inlet port 260 is fluidly isolated from the second outlet port 272, such that when the reaction chamber 120 is filled beyond its fixed volume, any excess contents are directed to the second waste chamber 148. However, the normally open valve 256 is movable (e.g., by applying a current to the valve 256) from the open position to a closed, or second, position when, for example, the reaction chamber 120 is no longer receiving the above-described contents and it is time to clean the reaction chamber 120. In this closed position, the inlet port 260 is fluidly coupled to the second outlet port 272 and the inlet port 260 is fluidly isolated from the first outlet port 268, such that the outlet 264 of the reaction chamber 120 is exposed to the atmosphere. In turn, air can flow into the reaction chamber 120, thereby facilitating the removal of contents from the reaction chamber 120.

The second column 124, also referred to herein as the desalting column 124, preferably takes the form of a size exclusion chromatography column that is located downstream of the valve 104 and is fluidly coupled to the satellite port 170 of the valve 104 via a conduit 276 of the plumbing. The second column 124 is thus arranged to receive the denatured, reduced, and alkylated polypeptides, the elution buffer solution 204, the alkylating agent, the reducing reagent, and the denaturing reagent (when one is used) from the reaction chamber 120. In this version, the second column 124 indirectly receives these materials from the reaction chamber 120. More particularly, these materials are moved from the reaction chamber 120 to the first holding coil 108, via the valve 104, when the valve 104 is in (or moved to) the fourth position (in which the central port 160 is fluidly coupled to the satellite port 167), and the valve 104 is moved to a seventh position in which the central port 160 is fluidly coupled to the satellite port 170, such that the materials move from the first holding coil 108 to the second column 124. In other versions, the second column 124 can directly receive these materials from the reaction chamber 120 or can indirectly receive these materials utilizing one or more different components and/or in a different order.

In any event, when the second column 124 receives the denatured, reduced, and alkylated polypeptides, the elution buffer solution 204, the alkylating agent, the reducing reagent, and the denaturing reagent (when one is used), the second column 124 is configured to separate the denatured, reduced, and alkylated polypeptides from the elution buffer solution 204, the alkylating agent, the reducing reagent, and the denaturing reagent (when used), which are ultimately moved to the third waste chamber 152. Thus, the second column 124 can be referred to herein as the desalting column 124. At the same time, the second column 124 is configured to buffer exchange the polypeptides into a desired buffer condition that allow the third column 128 to perform the functionality described below.

In this version, the system 100 further includes a second normally open valve 280 that is fluidly coupled to and located between the second column 124 and the third column 128. The normally open valve 280 has an inlet port 284 that is fluidly connected to an outlet 288 of the second column 124, a first outlet port 292 that is fluidly connected to the third waste chamber 152, and a second outlet port 296 that is fluidly connected to the third column 128. The normally open valve 280 normally operates in an open, or first, position when the system 100 is in operation. In this open position, the inlet port 284 is fluidly coupled to the first outlet port 292 and the inlet port 284 is fluidly isolated from the second outlet port 296, such that the elution buffer solution 204, the alkylating agent, the reducing reagent, and the denaturing reagent are directed to the third waste chamber 152. However, the normally open valve 280 is movable (e.g., by applying a current to the valve 280) from the open position to a closed, or second, position when it is desired to move the desalted polypeptides from the second column 124 to the third column 128, which is located downstream of the second column 124. In this closed position, the inlet port 284 is fluidly coupled to the second outlet port 296 and the inlet port 284 is fluidly isolated from the first outlet port 292, such that the second column 124 is fluidly coupled to the third column 128, such that the desalted can pass from the second column 124 to the third column 128. In this version, the third column 128 includes a proteolytic enzyme (e.g., an endopeptidase selected from the group consisting of trypsin, chymotrypsin, elastase, thermolysin, pepsin, glutamyl endopeptidase, neprilysin, Lys-C protease, and Staphylococcus aureus V8 protease), such that the third column 128 can be referred to herein as a proteolytic enzyme column. In any case, the third column 128 digests the desalted polypeptides obtained from the second column 124.

After the polypeptides have been digested in the third column 128, the digested polypeptides can be moved to the analytical device 156, which can, for example, take the form of a liquid chromatography device, a high-performance liquid chromatography device, an ultra high-performance liquid chromatography device, a mass spectrometry device, a glycan analysis device, another analysis device, or a combination thereof. In this version, the analytical device 156 is located downstream of the third column 128 and is fluidly coupled to the third column 128 via a conduit 298 of the plumbing. Thus, in this version, the digested polypeptides can be automatically moved to the analytical device 156 for analysis (e.g., for quantification and separation). In other versions, however, the analytical device 156 is not be part of the system 100 (e.g., not fluidly coupled to the third column 128), in which case the digested polypeptides can be moved to the analytical device 156 in a different manner (e.g., manually).

As briefly noted above, the system 100 also includes the controller 132, which in this version is communicatively coupled or connected to various components of the system 100 to monitor and facilitate or direct the above-described operation of the system 100 by transmitting signals (e.g., control signals, data) to and receiving signals (e.g., data) from the various components of the system 100. The controller 132 can be located immediately adjacent the other components of the system 100 (e.g., in the same environment as the system 100) or can be remotely located from the other components of the system 100. As illustrated, the controller 132 is communicatively coupled or connected to the multi-port valve 104 via a communication network 300, the pump 140 via a communication network 328, the analytical device 156 via a communication network 332, the heating element 236 via a communication network 340, the first normally open valve 256 via a communication network 344, and the second normally open valve 280 via a communication network 348. In other versions, the controller 132 can be communicatively coupled or connected to more or less components of the system 100, e.g., the first holding coil 108, the first column 112, the second holding coil 116, the reaction chamber 120, the second column 124, the third column 128, the vessel 136, the three-way valve 228, and/or the cooling vessel 240.

As used herein, the phrases “communicatively coupled” and “connected” are defined to mean directly coupled or connected to or indirectly coupled or connected through one or more intermediate components. Such intermediate components can include hardware and/or software-based components. It is appreciated that the networks 300-348 can be wireless networks, wired networks, or combinations of a wired and a wireless network (e.g., a cellular telephone network and/or 802.11x compliant network), and can include a publicly accessible network, such as the Internet, a private network, or a combination thereof. The type and configuration of the networks 300-348 is implementation dependent, and any type of communications networks which facilitate the described communications between the controller 132 and the components of the system 100, available now or later developed, can be used.

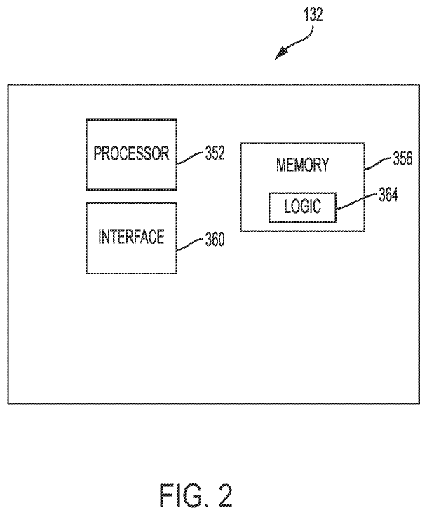

As shown in FIG. 2, the controller 132 includes a processor 352, a memory 356, a communications interface 360, and computing logic 364. The processor 352 can be a general processor, a digital signal processor, an application-specific integrated circuit (ASIC), field programmable gate array, graphics processing unit, analog circuit, digital circuit, or any other known or later developed processor. The processor 352 operates pursuant to instructions in the memory 356. The memory 356 can be a volatile memory or a non-volatile memory. The memory 356 can include one or more of a read-only memory (ROM), random-access memory (RAM), a flash memory, an electronic erasable program read-only memory (EEPROM), or other type of memory. The memory 356 can include an optical, magnetic (hard drive), or any other form of data storage device.

The communications interface 360 is provided to enable or facilitate electronic communication between the controller 132 and the components of the refrigeration system 100 via the networks 300-348. The communications interface 360 can be or include, for example, one or more universal serial bus (USB) ports, one or more Ethernet ports, and/or one or more other ports or interfaces. The electronic communication can occur via any known communications protocol, including, by way of example, USB, RS-232, RS-485, WiFi, Bluetooth, and/or any other suitable communications protocol.

The logic 364 generally includes one or more control routines and/or one or more sub-routines embodied as computer-readable instructions stored on the memory 356. The control routines and/or sub-routines can perform PID (proportional-integral-derivative), fuzzy logic, nonlinear, or any other suitable type of control. The processor 352 generally executes the logic 364 to perform actions related to the operation of the system 100.

Generally speaking, the logic 364, when executed, causes the processor 352 to control components of the system 100, particularly the multi-port valve 104, the pump 140, the heat element 236, the first and second normally open valves 256, 280, and the analytical device 156, such that the system 100 operates in the desired manner discussed herein. More particularly, the logic 364 can, when executed, cause the processor 352 to (i) move the multi-port valve 104 to or between any of the positions described herein, thereby fluidly coupling various components of the system 100 as described above, (ii) control the pump 140 (e.g., cause the pump 140 to obtain and output the elution buffer solution 204 or the denaturing reagent 208), (iii) control the heating element 236 (when employed in the system 100) to selectively apply heat to the reaction chamber 120 (and the contents thereof), (iv) control the first normally open valve 256, (v) control the second normally open valve 280, (vi) control the analytical device 156, and perform other desired functionality.

When, for example, it is desired to perform a real-time assay of a sample of a product containing polypeptides, the logic 364 is executable by the processor 352 to position the valve 104 in the first position described above, move the sample of the product from the vessel 136 to the first holding coil 108 via the conduit 180, the port 164, the port 160, position the valve 104 in the second position described above, and move the sample from the first holding coil 108 to and through the polypeptide-binding column 112 via the conduit 184, the ports 160, 164, and the conduit 188. In turn, substantially all of the polypeptides in the sample bind to the column 112, such that the polypeptides in the sample are separated from the remainder of the sample, which passes to the first waste chamber 144 via the conduits 220, 224 and the second holding coil 116.

The logic 364 is further executable by the processor 352 to cause the pump 140 to obtain the elution buffer solution 204 from the first buffer source 196 and output the elution buffer solution 204 to the first holding coil 108 via the conduit 192. In some cases, the pump 140 can need to be moved from the second position to the first position (to fluidly couple the pump 140 with the first buffer source 196), but in other cases, the pump 140 can already be in the first position. In any case, the logic 364 is executable by the processor 352 to move the elution buffer solution 204 from the first holding coil 108 to and through the first column 112 and to the second holding coil 116, via the conduit 184, the ports 160, 165, and the conduits 188, 220, and 224. In this manner, the elution buffer solution 204 elutes substantially all of the polypeptides bound to the first column 112, and an elution/polypeptide mixture including the elution buffer solution 204 and the eluted polypeptides flows from the first column 112 to the second holding coil 116.

The logic 364 is further executable by the processor 352 to move the valve 104 to the third position described above, move the elution/polypeptide mixture from the second holding coil 116 to the first holding coil 108, via the conduits 224, 216, the ports 166, 160, and the conduit 184, move the valve 104 to the fourth position described above, and move the elution/polypeptide mixture from the first holding coil 108 to the reaction chamber 120, via the conduit 184, the ports 160, 167, and the conduit 232. In turn, the polypeptides in the elution/polypeptide mixture are incubated in the reaction chamber 120 with the denaturing reagent 208 and/or in the presence of heat applied by the heating element 236, which thereby denatures the polypeptides.

The logic 364 is further executable by the processor 352 to move the valve 104 to the fifth position described above, move the reducing reagent from the first chamber 242 of the cooling vessel 240 to the first holding coil 108 via the conduit 244, the ports 168, 160, and the conduit 184, move the valve 104 back to the fourth position, and move the reducing reagent from the first holding coil 108 to the reaction chamber 120, via the conduit 184, the ports 160, 167, and the conduit 232. Upon reaching the reaction chamber 120, the reducing reagent cleaves disulfide bond crosslinks, which thereby reduces the denatured polypeptides in the reaction chamber 120.

The logic 364 is further executable by the processor 352 to move the valve 104 to the sixth position described above, move the alkylating agent from the second chamber 248 of the cooling vessel 240 to the first holding coil 108 via the conduit 252, the ports 169, 160, and the conduit 184, move the valve 104 back to the fourth position, and move the alkylating agent from the first holding coil 108 to the reaction chamber 120, via the conduit 184, the ports 160, 167, and the conduit 232. Upon reaching the reaction chamber 120, the alkylating agent alkylates sulfhydryls in the reaction chamber 120, which thereby alkylates the denatured and reduced polypeptides in the reaction chamber 120.

The logic 364 is further executable by the processor 352 to move the valve 104 to the fourth position (if not already there), move the denatured, reduced, and alkylated polypeptides, the elution buffer solution 204, the alkylating agent, the reducing reagent, and the denaturing reagent (when used) from the reaction chamber 120 to the first holding coil 108, via the conduit 232, the ports 167, 160, and the conduit 184, move the valve 104 to the seventh position described above, and move the denatured, reduced, and alkylated polypeptides, the elution buffer solution 204, the alkylating agent, the reducing reagent, and the denaturing reagent (when used) from the first holding coil 108 to the desalting column 124 via the conduit 184, the ports 160, 170, and the conduit 276. In turn, the desalting column 124 separates the denatured, reduced, and alkylated polypeptides from the elution buffer solution 204, the alkylating agent, the reducing reagent, and the denaturing reagent, which are passed or move to the third waste chamber 152.

At some point after the denatured, reduced, and alkylated polypeptides, the elution buffer solution 204, the alkylating agent, the reducing reagent, and the denaturing reagent (when used) are moved from the reaction chamber 120 to the first holding coil 108, the logic 364 is further executable by the processor 352 to move the normally open valve 256 from its open, first position, wherein the outlet 264 of the chamber 120 is fluidly coupled to the second waste chamber 148 so as to direct contents that will not fit in the reaction chamber 120 (as a result of it being filled beyond its fixed volume) to the second waste chamber 148, to its closed, second position, wherein the outlet 264 is fluidly coupled to atmosphere, such that air can flow into the reaction chamber 120, thereby facilitating the removal of contents from the reaction chamber 120. The normally open valve 256 can return to the open, first position immediately after the reaction chamber 120 has been emptied or can return to the open, first position at a later point in time.

After the desalting column 124 separates the denatured, reduced, and alkylated polypeptides from the other materials, the logic 364 is further executable by the processor 352 to move the normally open valve 280 from its open, first position, wherein the inlet port of the valve 280 is fluidly coupled to the third waste chamber 152, to its closed, second position, wherein the inlet port of the valve 280 is fluidly coupled to the proteolytic enzyme column 128. In turn, the logic 364 is executable by the processor 352 to move the desalted (or separated) polypeptides from the desalting column 124 to the proteolytic enzyme column 128, which digests the desalted polypeptides.

After the polypeptides have been digested, the logic 364 is, at least in this version, further executable by the processor 352 to move the digested polypeptides from the proteolytic enzyme column 128 to the analytical device 156 for analysis of the polypeptides, and to cause the analytical device 156 to perform the desired analysis. As an example, the logic 364 can, when executed by the processor 352, cause the analytical device 156 to separate and quantify the polypeptides.

In other versions, the logic 364 can, when executed by the processor 352, cause additional, less, and/or different functionality to be performed. As an example, the logic 364, when executed by the processor 352, may not move the digested polypeptides from the column 128 to the analytical device 156 or cause the analytical device 156 to perform the desired analysis. Moreover, in other versions, the logic 364 can be executed by the processor 352 in a different order than described herein. Finally, it is appreciated that the logic 364 can be executed by the processor 352 any number of different times, as the system 100 can be used to perform real-time analyses of multiple samples (from the same product and/or from a different product).

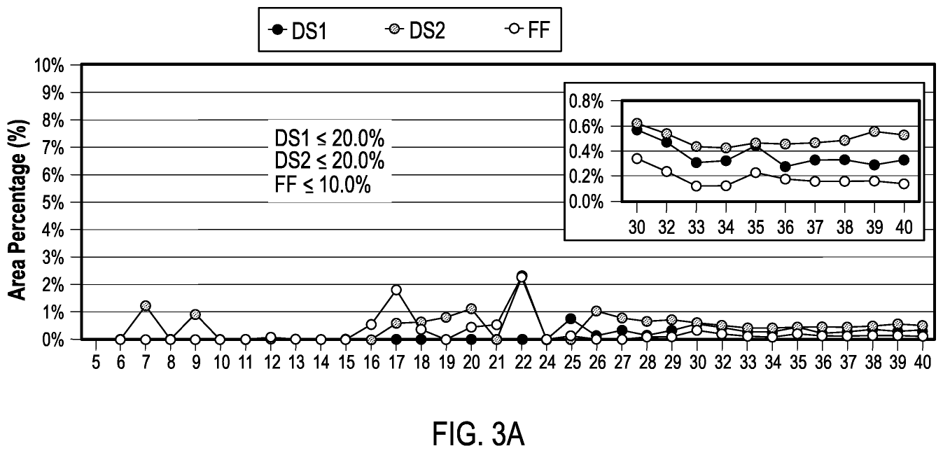

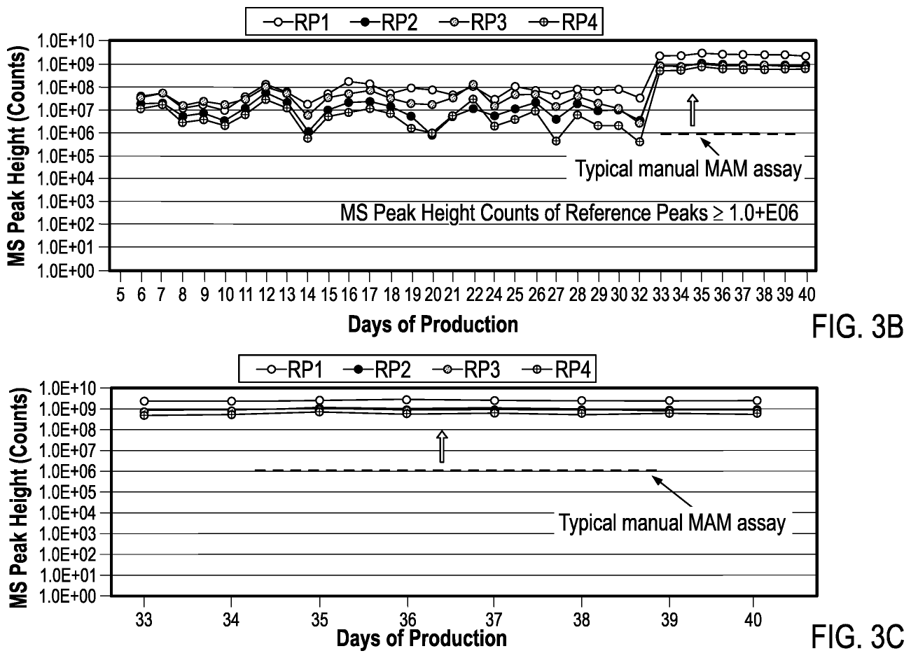

FIGS. 3A-3C illustrate the results of an online and real-time MAM assay study designed to monitor the effectiveness of the system 100 in preparing a sample of a product containing a Bispecific T-cell Engager (BiTE®) molecule. In particular, the study monitored the effectiveness of the system 100 over a production run of 40 days. The study began monitoring and collecting CQA data, such as area percentage for 2 deamidation sites, DS1 and DS2, and the frequency of a fragmentation, FF, illustrated in FIG. 3A, and MS peak height (expressed as a number of ion counts) for four reference peptides RP1, RP2, RP3, and RP4, illustrated in FIGS. 3B and 3C, on day 6 of the 40-day production run. As illustrated in FIG. 3A, the system 100 capably and effectively performed the intended functionality discussed herein over the entire duration of the 40-day production run, and, as illustrated in FIGS. 3B and 3C, the CQA data collected between day 6 and day 40 was substantially consistent, i.e., there was no significant change in product quality over time, and the product quality actually increased after day 32, thereby demonstrating the robustness of the system 100 in automatically preparing the sample. Indeed, as illustrated in FIG. 3C, the CQA data collected for RP1, RP2, RP3, and RP4 during that time-period was better than the CQA data obtained during a typical manual MAM assay.

### Therapeutic polypeptides

Proteins, including those that bind to one or more of the following, can be useful in the disclosed devices and methods. These include CD proteins, including CD3, CD4, CD8, CD19, CD20, CD22, CD30, and CD34; including those that interfere with receptor binding. HER receptor family proteins, including HER2, HER3, HER4, and the EGF receptor. Cell adhesion molecules, for example, LFA-I, MoI, pl50, 95, VLA-4, ICAM-I, VCAM, and alpha v/beta 3 integrin. Growth factors, such as vascular endothelial growth factor (“VEGF”), growth hormone, thyroid stimulating hormone, follicle stimulating hormone, luteinizing hormone, growth hormone releasing factor, parathyroid hormone, Mullerian-inhibiting substance, human macrophage inflammatory protein (MIP-I-alpha), erythropoietin (EPO), nerve growth factor, such as NGF-beta, platelet-derived growth factor (PDGF), fibroblast growth factors, including, for instance, aFGF and bFGF, epidermal growth factor (EGF), transforming growth factors (TGF), including, among others, TGF-α and TGF-β, including TGF-β1, TGF-β2, TGF-β3, TGF-β4, or TGF-β 5, insulin-like growth factors-I and -II (IGF-I and IGF-II), des(1-3)-IGF-I (brain IGF-I), and osteoinductive factors. Insulins and insulin-related proteins, including insulin, insulin A-chain, insulin B-chain, proinsulin, and insulin-like growth factor binding proteins. Coagulation and coagulation-related proteins, such as, among others, factor VIII, tissue factor, von Willebrands factor, protein C, alpha-1-antitrypsin, plasminogen activators, such as urokinase and tissue plasminogen activator (“t-PA”), bombazine, thrombin, and thrombopoietin; (vii) other blood and serum proteins, including but not limited to albumin, IgE, and blood group antigens. Colony stimulating factors and receptors thereof, including the following, among others, M-CSF, GM-CSF, and G-CSF, and receptors thereof, such as CSF-1 receptor (c-fms). Receptors and receptor-associated proteins, including, for example, flk2/flt3 receptor, obesity (OB) receptor, LDL receptor, growth hormone receptors, thrombopoietin receptors (“TPO-R,” “c-mpl”), glucagon receptors, interleukin receptors, interferon receptors, T-cell receptors, stem cell factor receptors, such as c-Kit, and other receptors. Receptor ligands, including, for example, OX40L, the ligand for the OX40 receptor. Neurotrophic factors, including bone-derived neurotrophic factor (BDNF) and neurotrophin-3, -4, -5, or -6 (NT-3, NT-4, NT-5, or NT-6). Relaxin A-chain, relaxin B-chain, and prorelaxin; interferons and interferon receptors, including for example, interferon-α, -β, and -γ, and their receptors. Interleukins and interleukin receptors, including IL-I to IL-33 and IL-I to IL-33 receptors, such as the IL-8 receptor, among others. Viral antigens, including an AIDS envelope viral antigen. Lipoproteins, calcitonin, glucagon, atrial natriuretic factor, lung surfactant, tumor necrosis factor-alpha and-beta, enkephalinase, RANTES (regulated on activation normally T-cell expressed and secreted), mouse gonadotropin-associated peptide, DNAse, inhibin, and activin. Integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, addressins, regulatory proteins, immunoadhesins, antibodies. Myostatins, TALL proteins, including TALL-I, amyloid proteins, including but not limited to amyloid-beta proteins, thymic stromal lymphopoietins (“TSLP”), RANK ligand (“OPGL”), c-kit, TNF receptors, including TNF Receptor Type 1, TRAIL-R2, angiopoietins, and biologically active fragments or analogs or variants of any of the foregoing.

Exemplary polypeptides and antibodies include Activase® (Alteplase); alirocumab, Aranesp® (Darbepoetin-alfa), Epogen® (Epoetin alfa, or erythropoietin); Avonex® (Interferon β-Ia); Bexxar® (Tositumomab); Betaseron® (Interferon-β); bococizumab (anti-PCSK9 monoclonal antibody designated as L1L3, sec U.S. Pat. No. 8,080,243); Campath® (Alemtuzumab); Dynepo® (Epoetin delta); Velcade® (bortezomib); MLN0002 (anti-α4β7 mAb); MLN1202 (anti-CCR2 chemokine receptor mAb); Enbrel® (etanercept); Eprex® (Epoetin alfa); Erbitux® (Cetuximab); evolocumab; Genotropin® (Somatropin); Herceptin® (Trastuzumab); Humatrope® (somatropin [rDNA origin] for injection); Humira® (Adalimumab); Infergen® (Interferon Alfacon-1); Natrecor® (nesiritide); Kineret® (Anakinra), Leukine® (Sargamostim); LymphoCide® (Epratuzumab); Benlysta™ (Belimumab); Metalyse® (Tenecteplase); Mircera® (methoxy polyethylene glycol-epoetin beta); Mylotarg® (Gemtuzumab ozogamicin); Raptiva® (efalizumab); Cimzia® (certolizumab pegol); Soliris™ (Eculizumab); Pexelizumab (Anti-C5 Complement); MEDI-524 (Numax®); Lucentis® (Ranibizumab); Edrecolomab (,Panorex®); Trabio® (lerdelimumab); TheraCim hR3 (Nimotuzumab); Omnitarg (Pertuzumab, 2C4); Osidem® (IDM-I); OvaRex® (B43.13); Nuvion® (visilizumab); Cantuzumab mertansine (huC242-DMl); NeoRecormon® (Epoetin beta); Neumega® (Oprelvekin); Neulasta® (Pegylated filgastrim, pegylated G-CSF, pegylated hu-Met-G-CSF); Neupogen® (Filgrastim); Orthoclone OKT3® (Muromonab-CD3), Procrit® (Epoctin alfa); Remicade® (Infliximab), Reopro® (Abciximab), Actemra® (anti-IL6 Receptor mAb), Avastin® (Bevacizumab), HuMax-CD4 (zanolimumab), Rituxan® (Rituximab); Tarceva® (Erlotinib); Roferon-A®-(Interferon alfa-2a); Simulect® (Basiliximab); Stelara™ (Ustekinumab); Prexige® (lumiracoxib); Synagis® (Palivizumab); 146B7-CHO (anti-IL15 antibody, see U.S. Pat. No. 7,153,507), Tysabri® (Natalizumab); Valortim® (MDX-1303, anti-B. anthracis Protective Antigen mAb); ABthrax™; Vectibix® (Panitumumab); Xolair® (Omalizumab), ETI211 (anti-MRSA mAb), IL-I Trap (the Fc portion of human IgGl and the extracellular domains of both IL-I receptor components (the Type I receptor and receptor accessory protein)), VEGF Trap (Ig domains of VEGFRl fused to IgGl Fc), Zenapax® (Daclizumab); Zenapax® (Daclizumab), Zevalin® (Ibritumomab tiuxetan), Zetia (ezetimibe), Atacicept (TACI-Ig), anti-α4β7 mAb (vedolizumab); galiximab (anti-CD80 monoclonal antibody), anti-CD23 mAb (lumiliximab); BR2-Fc (huBR3/huFc fusion protein, soluble BAFF antagonist); Simponi™ (Golimumab); Mapatumumab (human anti-TRAIL Receptor-1 mAb); Ocrelizumab (anti-CD20 human mAb); HuMax-EGFR (zalutumumab); M200 (Volociximab, anti-α5β1 integrin mAb); MDX-010 (Ipilimumab, anti-CTLA-4 mAb and VEGFR-I (IMC-18F1); anti-BR3 mAb; anti-C. difficile Toxin A and Toxin B C mAbs MDX-066 (CDA-I) and MDX-1388); anti-CD22 dsFv-PE38 conjugates (CAT-3888 and CAT-8015); anti-CD25 mAb (HuMax-TAC); anti-TSLP antibodies; anti-TSLP receptor antibody (U.S. Pat. No. 8,101,182); anti-TSLP antibody designated as A5 (U.S. Pat. No. 7,982,016); (anti-CD3 mAb (NI-0401); Adecatumumab (MT201, anti-EpCAM-CD326 mAb); MDX-060, SGN-30, SGN-35 (anti-CD30 mAbs); MDX-1333 (anti-IFNAR); HuMax CD38 (anti-CD38 mAb); anti-CD40L mAb; anti-Cripto mAb; anti-CTGF Idiopathic Pulmonary Fibrosis Phase I Fibrogen (FG-3019); anti-CTLA4 mAb; anti-eotaxinl mAb (CAT-213); anti-FGF8 mAb; anti-ganglioside GD2 mAb; anti-sclerostin antibodies (sec, U.S. Pat. No. 8,715,663 or U.S. Pat. No. 7,592,429) anti-sclerostin antibody designated as Ab-5 (U.S. Pat. No. 8,715,663 or U.S. Pat. No. 7,592,429); anti-ganglioside GM2 mAb; anti-GDF-8 human mAb (MYO-029); anti-GM-CSF Receptor mAb (CAM-3001); anti-HepC mAb (HuMax HepC); MEDI-545, MDX-1103 (anti-IFNα mAb); anti-IGFIR mAb; anti-IGF-IR mAb (HuMax-Inflam); anti-IL12/IL23p40 mAb (Briakinumab); anti-IL-23p19 mAb (LY2525623); anti-IL13 mAb (CAT-354); anti-IL-17 mAb (AIN457); anti-IL2Ra mAb (HuMax-TAC); anti-IL5 Receptor mAb; anti-integrin receptors mAb (MDX-018, CNTO 95); anti-IPIO Ulcerative Colitis mAb (MDX-1100); anti-LLY antibody; BMS-66513; anti-Mannose Receptor/hCGβ mAb (MDX-1307); anti-mesothelin dsFv-PE38 conjugate (CAT-5001); anti-PDlmAb (MDX-1 106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; anti-ZP3 mAb (HuMax-ZP3); and an amyloid-beta monoclonal antibody comprising sequences, SEQ ID NO:8 and SEQ ID NO:6 (U.S. Pat. No. 7,906,625).

Examples of antibodies suitable for the methods and pharmaceutical formulations include the antibodies shown in Table 1. Other examples of suitable antibodies include infliximab, bevacizumab, cetuximab, ranibizumab, palivizumab, abagovomab, abciximab, actoxumab, adalimumab, afelimomab, afutuzumab, alacizumab, alacizumab pegol, ald518, alemtuzumab, alirocumab, altumomab, amatuximab, anatumomab mafenatox, anrukinzumab, apolizumab, arcitumomab, aselizumab, altinumab, atlizumab, atorolimiumab, tocilizumab, bapincuzumab, basiliximab, bavituximab, bectumomab, belimumab, benralizumab, bertilimumab, besilesomab, bevacizumab, bezlotoxumab, biciromab, bivatuzumab, bivatuzumab mertansine, blinatumomab, blosozumab, brentuximab vedotin, briakinumab, brodalumab, canakinumab, cantuzumab mertansine, cantuzumab mertansine, caplacizumab, capromab pendetide, carlumab, catumaxomab, cc49, cedelizumab, certolizumab pegol, cetuximab, citatuzumab bogatox, cixutumumab, clazakizumab, clenoliximab, clivatuzumab tetraxetan, conatumumab, crenezumab, cr6261, dacetuzumab, daclizumab, dalotuzumab, daratumumab, demcizumab, denosumab, detumomab, dorlimomab aritox, drozitumab, duligotumab, dupilumab, ecromeximab, eculizumab, edobacomab, edrecolomab, falizumab, efungumab, elotuzumab, elsilimomab, enavatuzumab, enlimomab pegol, enokizumab, enoticumab, ensituximab, epitumomab cituxetan, epratuzumab, erenumab, erlizumab, ertumaxomab, etaracizumab, etrolizumab, evolocumab, exbivirumab, fanolesomab, faralimomab, farletuzumab, fasinumab, fbta05, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gemtuzumab ozogamicin, gevokizumab, girentuximab, glembatumumab vedotin, golimumab, gomiliximab, gs6624, ibalizumab, ibritumomab tiuxetan, icrucumab, igovomab, imciromab, imgatuzumab, inclacumab, indatuximab ravtansine, infliximab, intetumumab, inolimomab, inotuzumab ozogamicin, ipilimumab, iratumumab, itolizumab, ixekizumab, keliximab, labetuzumab, lebrikizumab, lemalesomab, lerdelimumab, lexatumumab, libivirumab, ligelizumab, lintuzumab, lirilumab, lorvotuzumab mertansine, lucatumumab, lumiliximab, mapatumumab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-cd3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, ocaratuzumab, ocrelizumab, odulimomab, ofatumumab, olaratumab, olokizumab, omalizumab, onartuzumab, oportuzumab monatox, oregovomab, orticumab, otelixizumab, oxelumab, ozanezumab, ozoralizumab, pagibaximab, palivizumab, panitumumab, panobacumab, parsatuzumab, pascolizumab, pateclizumab, patritumab, pemtumomab, perakizumab, pertuzumab, pexelizumab, pidilizumab, pintumomab, placulumab, ponezumab, priliximab, pritumumab, PRO 140, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samalizumab, sarilumab, satumomab pendetide, secukinumab, sevirumab, sibrotuzumab, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, telimomab aritox, tenatumomab, tefibazumab, teneliximab, teplizumab, teprotumumab, tezepelumab, TGN1412, tremelimumab, ticilimumab, tildrakizumab, tigatuzumab, TNX-650, tocilizumab, toralizumab, tositumomab, tralokinumab, trastuzumab, TRBS07, tregalizumab, tucotuzumab celmoleukin, tuvirumab, ublituximab, urelumab, urtoxazumab, ustekinumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, zolimomab aritox.

Antibodies also include adalimumab, bevacizumab, blinatumomab, cetuximab, conatumumab, denosumab, eculizumab, erenumab, evolocumab, infliximab, natalizumab, panitumumab, rilotumumab, rituximab, romosozumab, tezepelumab, and trastuzumab, and antibodies selected from Table 1.

TABLE 1
Examples of therapeutic antibodies

| | | | HC Type | | | LC | HC |
|---|---|---|---|---|---|---|---|
| Target | | Vis- | (in- | | | SEQ | SEQ |
| (informal | Conc. | cosity | cluding | LC | | ID | ID |
| name) | (mg/ml) | (cP) | allotypes) | Type | pI | NO | NO |
| anti- | 142.2 | 5.0 | IgG1 (f) | Kappa | 9.0 | 1 | 2 |
| amyloid | | | (R; EM) | | | | |
| GMCSF | 139.7 | 5.6 | IgG2 | Kappa | 8.7 | 3 | 4 |
| (247) | | | | | | | |
| CGRPR | 136.6 | 6.3 | IgG2 | Lambda | 8.6 | 5 | 6 |
| RANKL | 152.7 | 6.6 | IgG2 | Kappa | 8.6 | 7 | 8 |
| Sclerostin | 145.0 | 6.7 | IgG2 | Kappa | 6.6 | 9 | 10 |
| (27H6) | | | | | | | |
| IL-1R1 | 153.9 | 6.7 | IgG2 | Kappa | 7.4 | 11 | 12 |
| Myostatin | 141.0 | 6.8 | IgG1 (z) | Kappa | 8.7 | 13 | 14 |
| | | | (K; EM) | | | | |
| B7RP1 | 137.5 | 7.7 | IgG2 | Kappa | 7.7 | 15 | 16 |
| Amyloid | 140.6 | 8.2 | IgG1 (za) | Kappa | 8.7 | 17 | 18 |
| | | | (K; DL) | | | | |
| GMCSF | 156.0 | 8.2 | IgG2 | Kappa | 8.8 | 19 | 20 |
| (3.112) | | | | | | | |
| CGRP | 159.5 | 8.3 | IgG2 | Kappa | 8.7 | 21 | 22 |
| (32H7) | | | | | | | |
| CGRP | 161.1 | 8.4 | IgG2 | Lambda | 8.6 | 23 | 24 |
| (3B6.2) | | | | | | | |
| PCSK9 | 150.0 | 9.1 | IgG2 | Kappa | 6.7 | 25 | 26 |
| (8A3.1) | | | | | | | |
| PCSK9 | 150.0 | 9.2 | IgG2 | Kappa | 6.9 | 27 | 28 |
| (492) | | | | | | | |
| CGRP | 155.2 | 9.6 | IgG2 | Lambda | 8.8 | 29 | 30 |
| Hepcidin | 147.1 | 9.9 | IgG2 | Lambda | 7.3 | 31 | 32 |
| TNFR | 157.0 | 10.0 | IgG2 | Kappa | 8.2 | 33 | 34 |
| p55) | | | | | | | |
| OX40L | 144.5 | 10.0 | IgG2 | Kappa | 8.7 | 35 | 36 |
| HGF | 155.8 | 10.6 | IgG2 | Kappa | 8.1 | 37 | 38 |
| GMCSF | 162.5 | 11.0 | IgG2 | Kappa | 8.1 | 39 | 40 |
| Glucagon | 146.0 | 12.1 | IgG2 | Kappa | 8.4 | 41 | 42 |
| R | | | | | | | |
| GMCSF | 144.5 | 12.1 | IgG2 | Kappa | 8.4 | 43 | 44 |
| (4.381) | | | | | | | |
| Sclerostin | 155.0 | 12.1 | IgG2 | Kappa | 7.8 | 45 | 46 |
| (13F3) | | | | | | | |
| CD-22 | 143.7 | 12.2 | IgG1 (f) | Kappa | 8.8 | 47 | 48 |
| | | | (R; EM) | | | | |
| INFgR | 154.2 | 12.2 | IgG1 (za) | Kappa | 8.8 | 49 | 50 |
| | | | (K; DL) | | | | |
| Ang2 | 151.5 | 12.4 | IgG2 | Kappa | 7.4 | 51 | 52 |
| TRAILR2 | 158.3 | 12.5 | IgG1 (f) | Kappa | 8.7 | 53 | 54 |
| | | | (R; EM) | | | | |
| EGFR | 141.7 | 14.0 | IgG2 | Kappa | 6.8 | 55 | 56 |
| IL-4R | 145.8 | 15.2 | IgG2 | Kappa | 8.6 | 57 | 58 |
| IL-15 | 149.0 | 16.3 | IgG1 (f) | Kappa | 8.8 | 59 | 60 |
| | | | (R; EM) | | | | |
| IGF1R | 159.2 | 17.3 | IgG1 (za) | Kappa | 8.6 | 61 | 62 |
| | | | (K; DL) | | | | |
| IL-17R | 150.9 | 19.1 | IgG2 | Kappa | 8.6 | 63 | 64 |
| Dkk1 | 159.4 | 19.6 | IgG2 | Kappa | 8.2 | 65 | 66 |
| (6.37.5) | | | | | | | |
| Sclerostin | 134.8 | 20.9 | IgG2 | Kappa | 7.4 | 67 | 68 |
| TSLP | 134.2 | 21.4 | IgG2 | Lambda | 7.2 | 69 | 70 |
| Dkk1 | 145.3 | 22.5 | IgG2 | Kappa | 8.2 | 71 | 72 |
| (11H10) | | | | | | | |
| PCSK9 | 145.2 | 22.8 | IgG2 | Lambda | 8.1 | 73 | 74 |
| GIPR | 150.0 | 23.0 | IgG1 (z) | Kappa | 8.1 | 75 | 76 |
| (2G10.006) | | | (K; EM) | | | | |
| Activin | 133.9 | 29.4 | IgG2 | Lambda | 7.0 | 77 | 78 |
| Sclerostin | 150.0 | 30.0 | IgG2 | Lambda | 6.7 | 79 | 80 |
| (2B8) | | | | | | | |
| Sclerostin | 141.4 | 30.4 | IgG2 | Kappa | 6.8 | 81 | 82 |
| c-fms | 146.9 | 32.1 | IgG2 | Kappa | 6.6 | 83 | 84 |
| α4β7 | 154.9 | 32.7 | IgG2 | Kappa | 6.5 | 85 | 86 |

* An exemplary concentration suitable for patient administration;
\hat{ }HC-antibody heavy chain; LC-antibody light chain.

Based on the foregoing description, it should be appreciated that the devices, systems, and methods described herein facilitate the performance of an assay of a sample substantially in real-time. Thus, the assay can be performed, and the desired result obtained, much more quickly than allowed by conventional processes.

It should also be appreciated that the devices, systems, and methods described herein allow the process of preparing the online, real-time assay using the system 100 to be easily monitored, which can in turn mitigate risk and extend a production run of the product. In particular, this process can be monitored by determining, e.g., using a controller such as the controller 132 and/or manually by an operator of the system 100, whether conditions in the system 100 are optimal, i.e., whether they satisfy a pre-determined performance threshold. As illustrated in FIGS. 3B and 3C, for example, MS Peak Height, expressed in ion counts, for the four reference peptides RP1, RP2, RP3, and RP4 can be obtained and compared against historical values for those reference peptides during the method qualification to determine whether conditions in the system 100 are optimal (and they are), such that the production run can be commenced, continued, or extended. However, when it is determined that the conditions in the system 100 are not optimal, at least one cell culture component can be adjusted until the conditions in the closed system are optimal. Examples of cell culture components that can be adjusted include, but are not limited to: pH, pressure, temperature, media flow (e.g., media flow rate, media feed rate), media content (including amino acids, nutrients, sugars, buffer), gassing strategy (e.g., mix of oxygen and carbon dioxide, gas rate), agitation (e.g., agitation rate), additives (e.g., metal additives, sugar additives), additive (e.g., anti-foam) feed rate, and perfusion rate. In some cases, only one cell culture component may need to be adjusted so that the conditions in the closed system are optimal, while in other cases, multiple cell culture components may need to be adjusted. Alternatively, when it is determined that the conditions are not optimal, or when the conditions in the system 100 are not optimal even after adjusting the at least one cell culture component, the controller and/or the operator of the system 100 may shut down the system 100.

In this manner, the devices, systems, and methods described herein also mitigate risk involved in the continued operation of the system 100 when conditions are not optimal or when it is otherwise undesirable to continue operation of the system 100. In particular, risk can be mitigated by determining (e.g., calculating or obtaining) process parameter and product quality data, e.g., pH, temperature, oxygen dissolution, cell viability, cell density, titer, aggregation, charge variant, glycosylation, etc., associated with the current operation of the system 100, determining whether the process parameter and product quality data satisfy a pre-determined risk threshold (determined before operation of the system 100 by a controller such as the controller 132 and/or responsive to input from the operator of the system 100), and then determining whether to continue, cease, or adjust operation of the system 100 based upon whether the process parameter and product quality data satisfy the pre-determined risk threshold. The pre-determined risk threshold may, for example, be determined by looking at (1) applying available process parameter data and product quality data to calculate average historical multi-variate (MV) data associated with the previous operation of the system 100 or some other similar system, and then (2) establishing the calculated average historical MV data as the threshold (the threshold may be calculated average historical MV data itself or some value or set of values based on the calculated average historical MV data. In one example, the pre-determined risk threshold may represent an acceptable deviation (e.g., three standard deviations) from the calculated average historical MV data. Alternatively or additionally, the pre-determined risk threshold may be determined based upon input from the operator of the system 100. In some cases, the system 100 may be shut down when the process parameter and product quality data associated with the current operation of the system 100 do not satisfy (e.g., exceed) the pre-determined risk threshold. In other cases, however, the system 100 may be adjusted when the process parameter and product quality data associated with the current operation of the system 100 do not satisfy (e.g., exceed) the pre-determined risk threshold or even when the process parameter and product quality data satisfy but are close to the pre-determined risk threshold.

Further, the Applicant has discovered that the devices, systems, and methods described herein also allow production runs using the system 100 to be extended. Conventional processes typically allow for 32 to 40-day production runs, at most. However, the Applicant has found that the devices, systems, and methods described herein allow for 50-80 if not 100 population doublings, i.e., approximately 50-80 if not 100 day production runs. Thus, more product can be obtained, all while operation of the system 100 is monitored to ensure that the product satisfies quality objectives and risk is mitigated.

Preferred embodiments of this disclosure are described herein, including the best mode or modes known to the inventors for carrying out the disclosure. Although numerous examples are shown and described herein, those of skill in the art will readily understand that details of the various embodiments need not be mutually exclusive. Instead, those of skill in the art upon reading the teachings herein should be able to combine one or more features of one embodiment with one or more features of the remaining embodiments. Further, it also should be understood that the illustrated embodiments are exemplary only, and should not be taken as limiting the scope of the disclosure. All methods described herein can be performed in any suitable order unless otherwise indicated herein or otherwise clearly contradicted by context. The use of any and all examples, or exemplary language (e.g., “such as”) provided herein, is intended merely to better illuminate the aspects of the exemplary embodiment or embodiments of the disclosure, and do not pose a limitation on the scope of the disclosure. No language in the specification should be construed as indicating any non-claimed element as essential to the practice of the disclosure.

1.-21. (canceled)

22. A closed system for performing an online, real-time assay, the system comprising:
a first holding coil fluidly arranged to receive a sample of a product containing polypeptides;
a multi-port valve fluidly coupled to and located downstream of the first holding coil;
a polypeptide binding column fluidly coupled to the multi-port valve and arranged to receive the sample from the first holding coil via a first port of the multi-port valve, the polypeptide binding column configured to bind the polypeptides from the sample,
a buffer source fluidly coupled to the multi-port valve and arranged to supply elution buffer solution to a second holding coil located downstream of the polypeptide binding column, such that the elution buffer solution is adapted to elute substantially all of the polypeptides from the polypeptide binding column;
a reaction chamber fluidly coupled to the multi-port valve and arranged downstream of the polypeptide binding column, the reaction chamber adapted to receive a mixture from the polypeptide binding column via a second port of the multi-port valve, the mixture comprising the elution buffer solution and the eluted polypeptides, wherein the polypeptides of the mixture are denatured in the reaction chamber, wherein the reaction chamber is arranged to receive a reducing reagent that cleaves disulfide bond crosslinks via the first holding coil and a third port of the multi-port valve, the reducing reagent reduces the denatured polypeptides, and wherein the reaction chamber is further arranged to receive an alkylating reagent that alkylates sulfhydryls via the first holding coil and the third port of the multi-port valve, wherein the alkylating reagent alkylates the denatured and reduced polypeptides in the reaction chamber;
a desalting column fluidly coupled to the multi-port valve and arranged to receive the denatured, reduced, and alkylated polypeptides, the elution buffer solution, and the alkylating reagent from the reaction chamber, the desalting column configured to separate the denatured, reduced, and alkylated polypeptides from the elution buffer solution, the reducing reagent, and the alkylating reagent; and
a proteolytic enzyme column fluidly coupled to and arranged downstream of the second polypeptide column to obtain the separated polypeptides from the desalting column, the proteolytic enzyme column configured to digest the desalted polypeptides.

23. The system of claim 22, further comprising a bioreactor configured to produce the product containing polypeptides, wherein the first holding coil is fluidly coupled to the bioreactor.

24. The system of claim 22, wherein the multi-port valve comprises a 12 satellite port and a central shared port valve.

25. The system of claim 22, wherein the polypeptide binding column is selected from the group consisting of a protein A column, a protein G column, a protein A/G column, a protein L column, an amino acid column, an avidin column, a streptavidin column, a carbohydrate bonding column, a carbohydrate column, a glutathione column, a heparin column, a hydrophobic interaction column, an immunoaffinity column, a nucleotide/coenzyme column, a specialty column, and an immobilized-metal affinity chromatography (IMAC) column.

26. The system of claim 22, further comprising a pump configured to pump the sample of the product from the bioreactor to the first holding coil.

27. The system of claim 22, wherein the reducing reagent is selected from the group consisting of dithiothreitol (DTT), glutathione, β-mercaptoethanol (B-ME), and tris(2-carboxyethyl) phosphine (TCEP).

28. The system of claim 22, wherein the alkylating reagent is indole-3-acetic acid (IAA).

29. The system of claim 22, wherein the desalting column is a size exclusion chromatography column.

30. The system of claim 22, wherein the proteolytic enzyme column comprises an proteolytic enzyme, and wherein the proteolytic enzyme is an endopeptidase.

31. The system of claim 30, wherein the endopeptidase is selected from the group consisting of trypsin, chymotrypsin, elastase, thermolysin, pepsin, glutamyl endopeptidase, neprilysin, Lys-C protease, and Staphylococcus aureus V8 protease.

32. The system of claim 22, wherein the reaction chamber is at least partially pre-filled with a denaturing reagent.

33. The system of claim 32, further comprising a pump configured to pump the denaturing reagent from a denaturing buffer source to the reaction chamber via the first holding coil.

34. The system of claim 32, wherein the denaturing reagent comprises a denaturing detergent or a chaotrope.

35. The system of claim 34, wherein the chaotrope is selected from the group consisting of urea, n-butanol, ethanol, guanidinium chloride, lithium perchlorate, lithium acetate, magnesium chloride, phenol, 2-propanol, and thiourea.

36. The system of claim 34, wherein the denaturing detergent is selected from the group consisting of sodium dodecyl sulfate (SDS), sodium cholate, sodium deoxycholate, sodium glycocholate, sodium taurocholate, sodium taurodeoxycholate, N-lauroylsarcosine, lithium dodecyl sulfate, hexadecyltrimethyl ammonium bromide (CTAB) and trimethyl(tetradecyl) ammonium bromide (TTAB).

37. The system of claim 36, wherein the denaturing detergent is SDS.

38. The system of claim 32, wherein the denaturing reagent comprises a heated fluid.

39. The system of claim 22, further comprising a heating element positioned immediately adjacent the reaction chamber, the heating element configured to apply heat to the reaction chamber.

40. The system of claim 39, wherein the heat has a temperature of between about 22° C. to about 120° C.

41. The system of claim 40, wherein the heat has a temperature of about 40° C.

42. The system of claim 22, further comprising a first valve fluidly coupled to and located downstream of the reaction chamber, wherein the first valve is operable in a first position, in which the first valve directs contents received from the first holding coil in excess of a volume of the reaction chamber to waste, and in a second position, in which the first valve directs air into the reaction chamber.

43. The system of claim 42, further comprising a controller communicatively coupled to the first valve to control a position of the first valve, wherein the controller moves the first valve from the first position to the second position prior to moving the denatured, reduced, and alkylated polypeptides, the elution buffer solution, the denaturing reagent, the reducing reagent, and the alkylating reagent from the reaction chamber to the desalting column.

44. The system of claim 22, further comprising a second valve fluidly coupled to and located between the desalting and proteolytic enzyme columns, wherein the second valve is operable in a first position, in which the second valve directs contents received from the desalting column to waste, and in a second position, in which the second valve directs contents received from the desalting column to the proteolytic enzyme column.

45. The system of claim 44, further comprising a controller communicatively coupled to the second valve to control a position of the second valve, wherein the controller moves the second valve from the first position to the second position after the desalting column separates the denatured, reduced, and alkylated polypeptides from the elution buffer solution, the denaturing reagent, the reducing reagent, and the alkylating reagent.

46. The system of claim 22, further comprising a controller communicatively coupled to the reaction chamber, the controller configured to maintain the reaction chamber at a temperature of approximately 40 degrees Celsius.

47. The system of claim 22, further comprising a controller communicatively coupled to the multi-port valve, the controller configured to selectively open and close the first, second, third, and fourth ports of the multi-port valve.

48.-77. (canceled)
